(19) United States
(12) Patent Application Publication
(10) Pub. No.: US 2026/0097239 A1
(43) Pub. Date: Apr. 9, 2026

(54) ULTRASONIC THERAPY PATCH FOR USE INSIDE A HUMAN BODY

(71) Applicant: Zhijun PENG, Foshan (CN)

(72) Inventor: Zhijun PENG, Foshan (CN)

(21) Appl. No.: 18/991,753

(22) Filed: Dec. 23, 2024

(30) Foreign Application Priority Data
Oct. 6, 2024 (CN) 202411387685X

(51) Int. Cl. A61N 7/00

(52) U.S. Cl. CPC A61N 7/00

(57) ABSTRACT

An ultrasonic therapy patch used inside a human body, including a biomaterial bottom layer film and a biomaterial surface layer film laminated to each other, and a micro ultrasonic vibration module wrapped between the biomaterial bottom layer film and the biomaterial surface layer film. The ultrasonic therapy patch can be used safely inside the human body due to the biomaterials which it is made of, so that a site to be treated is directly covered by the patch and receives ultrasonic waves therapy directly from the patch.

### BACKGROUND OF THE INVENTION

The present invention relates to the field of ultrasonic therapy devices for a human body, and in particular to an ultrasonic therapy patch for use inside a human body.

An ultrasonic therapy device makes use of ultrasonic waves as the means for therapeutic treatment, wherein the ultrasonic waves outside the body are focused on a body part inside which pathological tissues are present; due to the ultrasonic waves, a temperature of the pathological tissues inside the body increases quickly, and a biological impact is resulted under mechanical reactions, thermal penetration and cavitation, thereby destroying the structures of the cells of the pathological tissues and causing the cells of the pathological tissues to wither. In addition, ultrasonic waves can promote blood circulation, accelerate metabolism, regenerate healthy tissues, and achieve a purpose of treating and curing the pathological tissues.

Prior art CN111544782A (application number 202010504927.4) titled “real-time positioning low-energy ultrasonic therapy device” has disclosed one of the ultrasonic therapy devices in the prior art; said prior art ultrasonic therapys mainly a treatment station and an ultrasonic irradiation probe; the treatment station ultrasonic therapys a support device, a control panel, an image display screen, and an ultrasonic generation device; the ultrasonic irradiation probe is connected to the ultrasonic generation device. During treatment, a medical personnel places the ultrasonic irradiation probe on the skin of a patient, and then the ultrasonic irradiation probe will radiate ultrasonic waves towards the patient and perform ultrasonic therapy on the body part to be treated. However, in practical application, the ultrasonic therapy device has the following deficiencies:

Firstly, this kind of ultrasonic therapy device is used externally outside the patient's body, therefore, the ultrasonic waves can only reach the site containing the pathological tissues after passing through the skin of the patient, and as a result, the ultrasonic waves cannot directly reach the site in concern. Moreover, the skin tissues can absorb part of the radiated ultrasonic energy, therefore the radiated ultrasonic energy that actually reaches the site is weakened. This reduces the destructive power to the cells of the pathological tissues and thus leads to slow therapeutic effect. If the intensity of the ultrasonic energy is increased, more significant side effects like damages to the skin tissues and cells will be resulted in spite of the fact that the pathological tissues can be killed under more intense ultrasonic energy; therefore, this approach of increasing the intensity of ultrasonic energy is still not preferred.

Secondly, only a professional medical personnel in a hospital knows how to operate the ultrasonic therapy device, and so the patient has to travel to the hospital frequently to receive regular treatment, thereby leading to extremely high costs in terms of both time and money.

Therefore, the applicant believes that it is highly necessary to design an ultrasonic therapy patch for use inside a human body, which can be implanted into the human body, enable the treatment to be performed directly on the site to be treated, and allow for regular treatment without the need to travel to the hospital frequently.

### BRIEF SUMMARY OF THE INVENTION

The present invention aims to solve the aforementioned problems and deficiencies by providing an ultrasonic therapy patch for use inside a human body. The ultrasonic therapy patch can be safely used in a human body due to the biomaterials that make the ultrasonic therapy patch, and thus can be directly applied to a site inside a body of a user to perform ultrasonic therapy on that site, so that ultrasonic waves can directly reach the site to be treated to ensure that cells of pathological tissues can be killed, and since the ultrasonic waves do not need to pass through the skin, the impact of the ultrasonic waves on the skin is significantly reduced. Moreover, a micro ultrasonic vibration module does not only perform ultrasonic vibration, but also has a wireless charging function and a wireless control function. The ultrasonic therapy patch can be used for a long time inside the human body once it is implanted into the human body, so that the patient can receive regular ultrasonic therapy at home without the need to travel to the hospital frequently, and the costs in terms of both time and money are greatly reduced.

The present invention is achieved as follows:

An ultrasonic therapy patch, comprising a biomaterial bottom layer film and a biomaterial surface layer film laminated to each other except for an area between the biomaterial bottom layer film and the biomaterial surface layer film in which a micro ultrasonic vibration module is provided between the biomaterial bottom layer film and the biomaterial surface layer film; the micro ultrasonic vibration module comprises a housing and a housing cover assembled to each other, wherein an accommodating cavity is defined between an enclosure formed by the housing and the housing cover; the micro ultrasonic vibration module also comprises an ultrasonic vibration assembly and a driving control assembly, both disposed in the accommodating cavity; the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect; the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

Preferably, the accommodating cavity is provided with a partition plate which partitions the accommodating cavity into a first working chamber and a second working chamber; the ultrasonic vibration assembly is disposed in the first working chamber, and the driving control assembly is disposed in the second working chamber.

Preferably, the second working chamber is stacked on the first working chamber.

Preferably, said at least one driving coil is disposed on the partition plate, and the slidable block is disposed on the housing cover.

Preferably, sensors electrically connected to the circuit board are further disposed at two ends of the housing respectively.

Preferably, transitional inclined planes are provided on peripheral sides of the housing.

Preferably, each of the biomaterial bottom layer film and the biomaterial surface layer film is provided with a plurality of air-permeable through holes.

Preferably, an adhesive layer is disposed on a bottom surface of the biomaterial bottom layer film, and a release paper is disposed on a bottom surface of the adhesive layer.

The present invention has the following beneficial effects: because a biomaterial bottom layer film and a biomaterial surface layer film are used where one is stacked upon another one, and a micro ultrasonic vibration module wrapped between the biomaterial bottom layer film and the biomaterial surface layer film is used in the present invention, the ultrasonic therapy patch can be implanted into the human body due to the biomaterials which it is made of, and after implantation, it has good biocompatibility, causes no foreign sensation, and does not result in adverse reactions, and thus can be safely used inside the human body. With such a structural design, the ultrasonic therapy patch can be directly applied to a site to be treated to directly perform ultrasonic therapy on that site, so that ultrasonic energy can directly reach the site to ensure that the ultrasonic energy is not weakened and cells of pathological tissues are killed. Moreover, by utilizing the ultrasonic cavitation effect generated by the ultrasonic waves, the ultrasonic therapy patch can also promote blood circulation, relieve soft tissues, reduce swelling, eliminate inflammation, improve the nutritional status of tissues, ablate a thrombus, and prevent the occurrence of thrombosis, thereby achieving an effect of assisting in self-repair of inner walls of blood vessels. In addition, the ultrasonic waves do not need to pass through the human skin, which can greatly reduce the impact of the ultrasonic waves on the human skin. Further, the micro ultrasonic vibration module of the present invention is provided with a driving control assembly, which does not only drive an ultrasonic vibration assembly to work, but also has a wireless charging function and a wireless control function. In this way, the ultrasonic therapy patch can be used for a long time inside the human body once being implanted, and a professional medical personnel can set parameters in a circuit board such as the intensity and duration of the ultrasonic energy being used, so that a user can receive regular ultrasonic therapy at home without the need to travel to the hospital frequently, and the costs in terms of both time and money are greatly reduced. Besides, drugs intake of the user can be greatly reduced due to the use of the present invention, and hence side effects of the drugs harmful to the body are greatly reduced, and the user's life may therefore by prolonged.

### BRIEF DESCRIPTION OF THE DRAWINGS

FIG. 1 is a schematic overall structural view of the present invention;

FIG. 2 is a schematic cross-sectional view at section A-A in FIG. 1 of the present invention;

FIG. 3 is a schematic exploded view of the present invention;

FIG. 4 is a schematic perspective view of a release paper and a biomaterial bottom layer film of the present invention;

FIG. 5 is a schematic perspective view of a micro ultrasonic vibration module of the present invention;

FIG. 6 is a first schematic diagram of an exploded structure of a micro ultrasonic vibration module of the present invention;

FIG. 7 is a second schematic diagram of an exploded structure of a micro ultrasonic vibration module of the present invention, where the housing cover and the components thereon are omitted;

FIG. 8 is a schematic cross-sectional view of a housing, a partition plate, and a housing cover of the present invention; and

FIG. 9 is a schematic diagram of a use state of the present invention when charging.

### DETAILED DESCRIPTION OF THE INVENTION

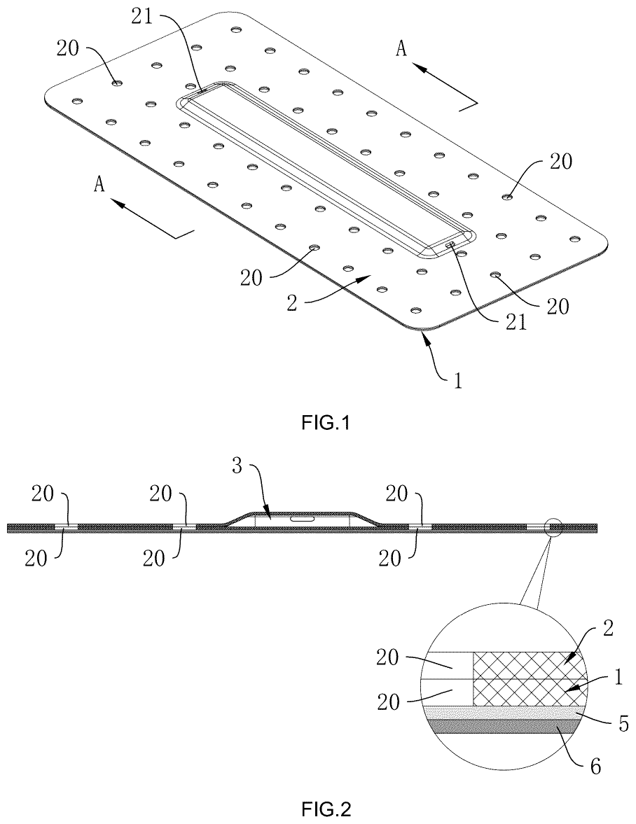

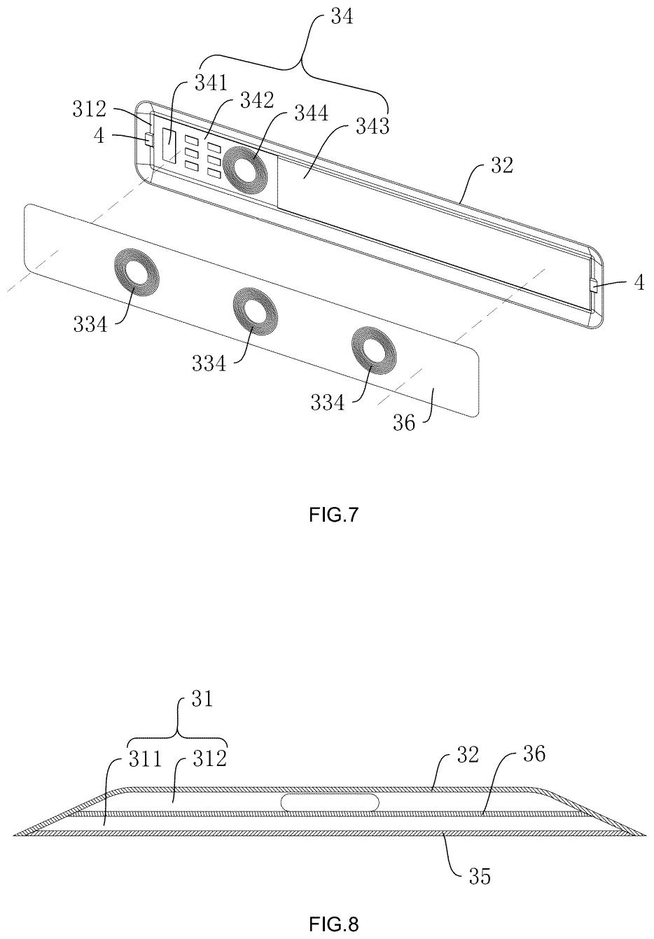

As shown in FIG. 1 and FIG. 2, an ultrasonic therapy patch for use inside a human body according to the present invention comprises a biomaterial bottom layer film 1 and a biomaterial surface layer film 2 laminated to each other except for an area between the biomaterial bottom layer film 1 and the biomaterial surface layer film 2 in which a micro ultrasonic vibration module 3 is provided between the biomaterial bottom layer film 1 and the biomaterial surface layer film 2. As shown in FIG. 5 to FIG. 7, the micro ultrasonic vibration module 3 comprises a housing 32 and a housing cover 35 assembled to each other, wherein an accommodating cavity 31 is defined between an enclosure formed by the housing 32 and the housing cover 35; the micro ultrasonic vibration module 3 also comprises an ultrasonic vibration assembly 33 and a driving control assembly 34, both disposed in the accommodating cavity 31. The ultrasonic vibration assembly 33 comprises a slidable block 332, resilient tabs 333 disposed at two ends of the slidable block 332 respectively, and at least one driving coil 334; at least one permanent magnet block 331 is provided on the slidable block 332; the resilient tabs 333 provides buffering and bouncing functions against the slidable block 332 as the slidable block 332 slides; said at least one driving coil 334 cooperates with said at least one permanent magnet block 331 to generate a magnetic driving effect. The driving control assembly 34 comprises a circuit board 342 on which a wireless communication module 341 is disposed, and a battery 343 and a wireless charging coil 344 which are electrically connected to the circuit board 342. Said at least one driving coil 334 is electrically connected to the circuit board 342 to drive the ultrasonic vibration assembly 33 to work.

The working principle of the ultrasonic vibration assembly 33 is as follows: The battery 343 supplies power to said at least one driving coil 334 through the circuit board 342, and a current flows into said at least one driving coil 334 to generate a magnetic field, which drives said at least one permanent magnet block 331 and thus the slidable block 332 to reciprocate, thereby generating high-frequency vibrations, which propagate through a medium such as gas, liquid, and solid to form ultrasonic waves.

Compared with the conventional eccentric motor, the ultrasonic vibration assembly 33 has the advantage of reducing, if not eliminating, motion inertia, and can therefore realize rapid start/stop of the reciprocation of the slidable block 332; further, the ultrasonic vibration assembly 33 does not require any space for eccentric rotation as in the case of using eccentric motor in the prior art, as a result, an overall thickness of the micro ultrasonic vibration module 3 can be smaller. Moreover, by providing the wireless communication module 341, a user can send on/off signals to the wireless communication module 341 through a wireless communication device, so that the circuit board 342 is remotely driven to control the start/stop of the ultrasonic vibration assembly 33. The wireless communication module 341 is an ultra-micro Bluetooth® communication module, WiFi® communication module, or the like. By utilizing a wireless communication module 341 to be linked to a device like a computer, a smartphone, or a tablet computer, remote operations, monitoring, and data transmissions of the micro ultrasonic vibration module 3 are realized.

In practical application, the biomaterial bottom layer film 1 and the biomaterial surface layer film 2 can be made by electronic skin with good air permeability, such as protein-based electronic skin PBES, or a silk fibroin-polyurethane SF-PU composite film, or a silk fibroin composite film SFCM.

In practical application, said at least one driving coil 334, the wireless charging coil 344, and the battery 343 are all electrically connected to the circuit board 342 through conducting wires, and the wireless communication module 341 is built-in to the circuit board 342.

Specifically, the accommodating cavity 31 may be a recess on the housing 32, or may be a recess on the housing cover 35.

As shown in FIG. 5 to FIG. 8, the accommodating cavity 31 is provided with a partition plate 36 for partitioning the accommodating cavity 31 into a first working chamber 311 and a second working chamber 312. The ultrasonic vibration assembly 33 is disposed in the first working chamber 311, and the driving control assembly 34 is disposed in the second working chamber 312. By partitioning the accommodating cavity 31 into two independent chambers, the ultrasonic vibration assembly 33 and the driving control assembly 34 can be prevented from interfering with each other, so that the operations of the two are more stable and reliable.

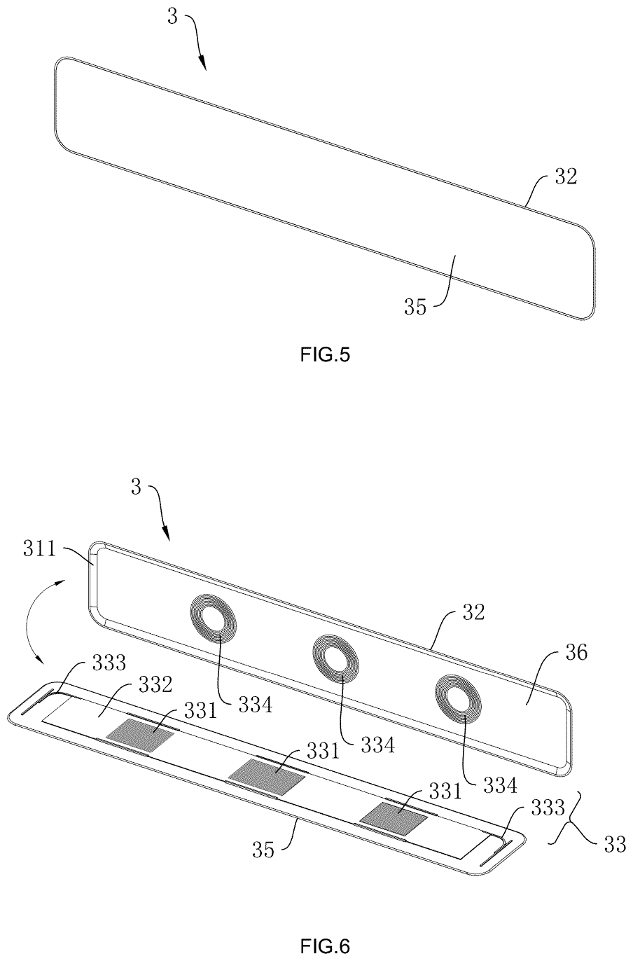

As shown in FIG. 6 to FIG. 9, the second working chamber 312 is stacked on the first working chamber 311. This enables the ultrasonic vibration assembly 33 to be disposed along a lengthwise direction of the micro ultrasonic vibration module 3, so that the vibration frequencies of various parts of the micro ultrasonic vibration module 3 are more balanced, and so the ultrasonic vibration treatment effect is better. Moreover, by positioning the second working chamber 312 above the first working chamber 311, the wireless charging coil 344 is positioned closer to a top surface of the housing 32, so that the wireless charging coil 344, as a receiving end of an external wireless charging device, can be closer to a transmitting end 100 of the external wireless charging device during wireless charging, thereby enabling the wireless transmission of electric energy to be more stable, reliable, and difficult to interrupt. Apart from using the aforementioned ultrasonic vibration assembly 33 as described above, the present invention can be alternatively implemented by using an ultrasonic transducer or an ultrasonic vibration motor in lieu of the ultrasonic vibration assembly 33. However, product performance is slightly poorer if the ultrasonic vibration motor is used.

As shown in FIG. 6, said at least one driving coil 334 is disposed on the partition plate 36, and the slidable block 332 is disposed on the housing cover 35. In this way, it is unnecessary to move the conducting wires out onto the housing cover 35, thereby facilitating assembly and preventing the conducting wires from affecting the sliding of the slidable block 332.

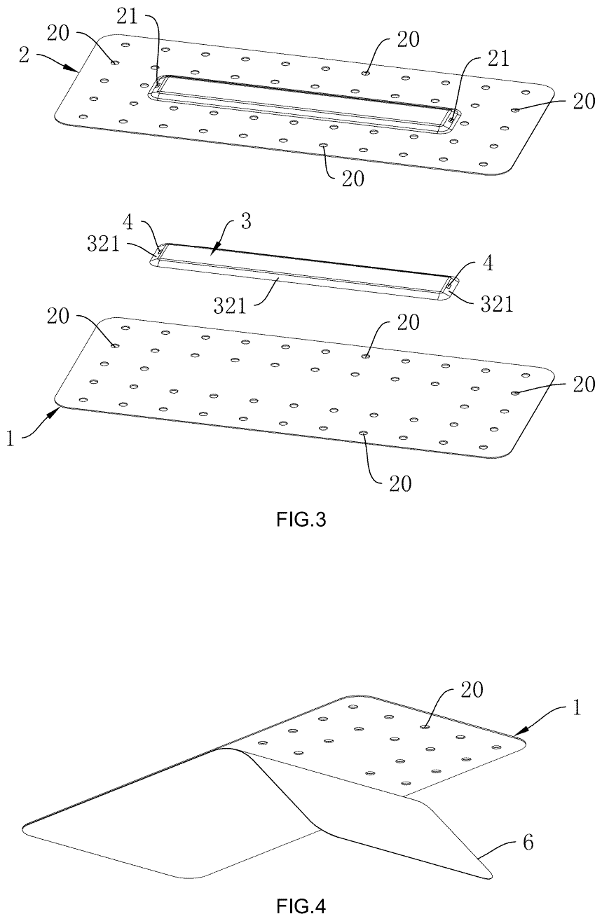

As shown in FIG. 3, sensors 4 electrically connected to the circuit board 342 are further disposed at two ends of the housing 32 respectively. Each of the sensors 4 can be a heart rate detection sensor, a blood oxygen detection sensor, a body temperature detection sensor, an image sensor mounted with a micro camera, or the like. In this way, it is convenient to observe and detect the health condition of the user, thereby increasing the usage functions of the ultrasonic therapy patch. When using two image sensors mounted with two micro cameras, anti-shielding through holes 21 corresponding to the two micro cameras are further provided on the biomaterial bottom layer film 1 or the biomaterial surface layer film 2.

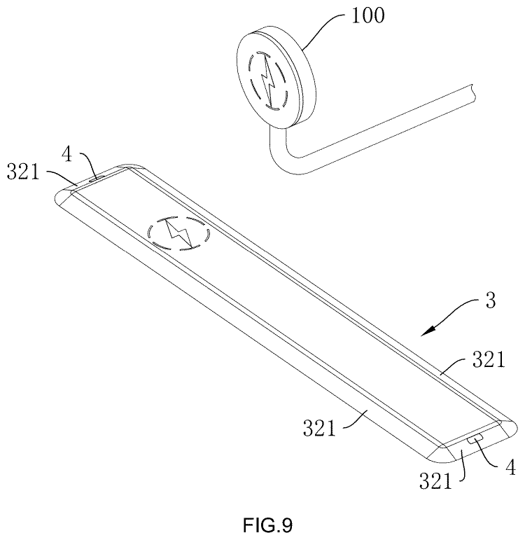

As shown in FIG. 9, transitional inclined planes 321 are further provided on peripheral sides of the housing 32. In this way, after the micro ultrasonic vibration module 3 is wrapped between the biomaterial bottom layer film 1 and the biomaterial surface layer film 2, excessively sharp edges and corners will not be formed on the biomaterial bottom layer film 1 and the biomaterial surface layer film 2, so that after the ultrasonic therapy patch is implanted into the human body, no obvious discomfort will be caused.

As shown in FIG. 3, each of the biomaterial bottom layer film 1 and the biomaterial surface layer film 2 is provided with a plurality of air-permeable through holes 20. Therefore, air permeability of the ultrasonic therapy patch can be further improved, and after the ultrasonic therapy patch is implanted into the human body, blood can be circulated through the air-permeable through holes 20, so that the ultrasonic therapy patch is more adaptable to the internal structural operations of a human body, and thereby reducing the adverse effect on the human body.

As shown in FIG. 2 and FIG. 4, an adhesive layer 5 is further disposed on a bottom surface of the biomaterial bottom layer film 1, and a release paper 6 is further disposed on a bottom surface of the adhesive layer 5. In this way, the connection stability between the ultrasonic therapy patch and the site of the human body to be treated can be improved, so that the ultrasonic therapy patch can be prevented from displacing, and the accuracy for the ultrasonic waves to reach the body part to be treated is ensured. By using the release paper 6, the adhesive layer 5 can be made less susceptible to contamination by impurities, thereby ensuring hygiene. In practical application, the adhesive layer 5 is composed of the commonly known and used WAB® biological adhesive manufactured by Jinan Shiji Bioadhesive Research Institute. If not using adhesive layer for fixation, suturing techniques commonly used in nowadays medical treatment can also be used to fix the ultrasonic therapy patch to the site of the human body to be treated.

What is claimed is:

1. An ultrasonic therapy patch, comprising a biomaterial bottom layer film and a
biomaterial surface layer film laminated to each other except for an area between the biomaterial bottom layer film and the biomaterial surface layer film in which a micro ultrasonic vibration module is provided between the biomaterial bottom layer film and the biomaterial surface layer film;
the micro ultrasonic vibration module comprises a housing and a housing cover assembled to each other, wherein an accommodating cavity is defined between an enclosure formed by the housing and the housing cover; the micro ultrasonic vibration module also comprises an ultrasonic vibration assembly and a driving control assembly, both disposed in the accommodating cavity;
the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect;
the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

2. The ultrasonic therapy patch of claim 1, wherein the accommodating cavity is provided with a partition plate which partitions the accommodating cavity into a first working chamber and a second working chamber; the ultrasonic vibration assembly is disposed in the first working chamber, and the driving control assembly is disposed in the second working chamber.

3. The ultrasonic therapy patch of claim 2, wherein the second working chamber is stacked on the first working chamber.

4. The ultrasonic therapy patch of claim 3, wherein said at least one driving coil is disposed on the partition plate, and the slidable block is disposed on the housing cover.

5. The ultrasonic therapy patch of claim 1, wherein sensors electrically connected to the circuit board are further disposed at two ends of the housing respectively.

6. The ultrasonic therapy patch of claim 1, wherein transitional inclined planes are provided on peripheral sides of the housing.

7. The ultrasonic therapy patch of claim 1, wherein each of the biomaterial bottom layer film and the biomaterial surface layer film is provided with a plurality of air-permeable through holes.

8. The ultrasonic therapy patch of claim 1, wherein an adhesive layer is disposed on a bottom surface of the biomaterial bottom layer film, and a release paper is disposed on a bottom surface of the adhesive layer.
